(19) United States
(12) Patent Application Publication
(10) Pub. No.: US 2003/0203362 A1
(43) Pub. Date: Oct. 30, 2003

(54) Species specific identification of spore-producing microbes using the gene sequence of small acid-soluble spore coat proteins for amplification based diagnostics

(76) Inventors: Jennifer C. Hunter-Cevera, Ellicott City, MD (US); Terrance Leighton, Lafayette, CA (US); Stan Goldman, Walnut Creek, CA (US); Pascal Longchamp, Palo Alto, CA (US); Nancy McKinney, La Honda, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/067,613

(22) Filed: Feb. 4, 2002

Related U.S. Application Data
(63) Continuation-in-part of application No. 09/590,759, filed on Jun. 8, 2000, now Pat. No. 6,472,155.
(60) Provisional application No. 60/192,206, filed on Mar. 27, 2000.
(60) Provisional application No. 60/138,167, filed on Jun. 8, 1999.

(51) Int. Cl. C12Q 1/68; C07H 21/04

(52) U.S. Cl. 435/6; 530/388.26; 536/23.7; 536/24.3

(57) ABSTRACT

The present invention relates to methods and compositions for the detection of Bacillus species such as Bacillus anthracis and Bacillus globigii as well as Clostridium perfringens. It relies on nucleic acid sequence differences in spore protein genes carried in the genomic sequence of these organisms.

### CROSS-REFERENCES TO RELATED APPLICATIONS

The present application is a Continuation-In-Part application (“CIP”) of U.S. application Ser. No. 09/590,759, filed Jun. 8, 2000, which claims priority benefit of U.S. Provisional Application No. 60/138,167, filed on Jun. 8, 1999, and U.S. Provisional Application No. 60/192,206, filed on Mar. 27, 2000. The aforementioned applications are explicitly incorporated herein by reference in their entirety and for all purposes.

### STATEMENT AS TO RIGHTS TO INVENTIONS MADE UNDER FEDERALLY SPONSORED RESEARCH OR DEVELOPMENT

This invention was made under work supported by the U.S. Department of Energy under DOE Contract No.: DE-AC03-76SF00098. The government has certain rights in this invention.

### REFERENCE TO A “SEQUENCE LISTING,” A TABLE, OR A COMPUTER PROGRAM LISTING APPENDIX SUBMITTED ON A COMPACT DISK.

Not Applicable

### BACKGROUND OF THE INVENTION

### FIELD OF THE INVENTION

The present invention relates to methods and compositions for the detection of Bacillus species such as Bacillus anthracis and Bacillus globigii as well as Clostridium perfingens. It relies on nucleic acid sequence differences in spore protein genes carried in the genomic sequence of these organisms.

The genus Bacillus is composed of rod-shaped, gram-positive, aerobic or (under some conditions) anaerobic bacteria widely found in soil and water. Most strains of Bacillus are not pathogenic for humans and only infect them incidentally in their role as soil organisms; a notable exception is Bacillus anthracis, which causes anthrax in humans and domestic animals. In addition to its role as a naturally occurring pathogen, Bacillus anthracis may also be used as a biological weapon. Because Bacillus organisms are widely distributed in the environment, and because they are very closely related genetically, there is need for a reliable method to distinguish species members in various types of samples.

Considerable efforts have been made to develop sensitive, species specific tests for these Bacillus microorganisms with only limited results; examples of such efforts follow:

Makino et al. describes a PCR-based test using primers to the cap region of plasmid pXO2 (codes for a protein essential for capsulation) which was amplified by PCR and probed with the B. anthracis capA DNA, Makino et al., “Direct Detection of Bacillus anthracis DNA in animals by Polymerase Chain Reaction”, J. Clin. Micro. March 1993, 31(3)547-51.

Keim et al. describes an AFLP (amplified-fragment length polymorphism) technique employing high resolution electrophoresis to examine restriction fragments of PCR products, Keim, P; Kalif, A; Schupp, J; Hill, K; Travis, SE; Richmond, K; Adair, D M; Hugh-Jones, M; Kuske, C R; Jackson, P., “Molecular evolution and diversity in Bacillus anthracis as detected by amplified fragment length polymorphism markers, Journal of Bacteriology, February 1997, 179(3):818-24.

Similarly, Andersen et al. describes a technique using arbitrarily primed (AP)-PCR “fingerprints” that detect variable numbers of repeats in different samples, Andersen et al. “Identification of a region of genetic variability among Bacillus anthracis strains and related species,” J. Bacteriology, January 1996, 178(2):377-84; Beyer, W; Glöckner, P; Otto, J; Böhm, R. “A nested PCR method for the detection of Bacillus anthracis in environmental samples collected from former tannery sites” Microbiological Research, May 1995, 150(2):179-86.

Reif et al. describes a PCR technique for identifying spores of B. anthracis. Primers specific for the capB region of plasmid pXO2 are used, Reif et al. “Identification of capsule forming Bacillus anthracis spores with the PCR and a novel dual-probe hybridization format,” Applied and Environmental Microbiology, May 1994, 60(5):1622-5.

Brightwell et al. describes a PCR reaction for detecting the presence of B. anthracis plasmid pXO2, Brightwell et al. “Development of internal controls for PCR detection of Bacillus anthracis” Molecular and Cellular Probes, (1998) 12, 367-377.

Jackson et al. describes PCR analysis using primers that detect the vrrA gene variable region on the B. anthracis chromosome, Jackson et al., P.N.A.S. 95:1224-1229, February 1998, “ PCR analysis of tissue samples from the 1979 Sverdlovsk anthrax victims: the presence of multiple Bacillus anthracis strains in different victims.”

Patral et al. describes a 277-bp long noncoding DNA fragment, Ba813, that was isolated from an avirulent Bacillus anthracis strain 7700 genomic library. Two oligonucleotides derived from the Ba813 sequence were used as primers in polymerase chain reaction tests on genomic DNA from 28 Bacillus anthracis and from 33 heterologous bacteria strains. A specific, 152-bp long DNA fragment was amplified only when Bacillus anthracis DNA was used as the target [Note: primers eventually demonstrated non-specific (McKinney, manuscript in preparation)], Patra, G; Sylvestre, P; Ramisse, V; Thaerasse, J; Guesdon, J L.” Isolation of a specific chromosomic DNA sequence of Bacillus anthracis and its possible use in diagnosis,” Fems Immunology and Medical Microbiology, Oct., 15, 1996 4 :223-31.

Most of the Bacillus anthracis PCR detection systems have targeted plasmid sequences, yet mobility of these genetic elements make them an unreliable detection target. (Battisti, L; Green, B D; Thorne, C B. Mating system for transfer of plasmids among Bacillus anthracis, Bacillus cereus, and Bacillus thuringiensis. Journal of Bacteriology, May 1985, 162(2):543-50. Green, B D; Battisti, L; Thorne, C B. Involvement of Tn4430 in transfer of Bacillus anthracis plasmids mediated by Bacillus thuringiensis plasmid pXO12. Journal of Bacteriology, January 1989, 171(1):104-13) Several of the PCR type assays to genomic sequences target non-gene-coding sequences, which often proves not to correlate well with species identity. The weaknesses in each of these systems underscores the need for a more reliable Bacillus identification method.

### SUMMARY OF THE INVENTION

It is an object of the present invention to provide methods and materials for specifically detecting and identifying members of the Bacillus family, especially Bacillus anthracis, to the exclusion of other members of the genus Bacillus, which are closely related. These methods and materials rely on the discovery that certain small acid-soluble protein (sasp) gene targets are present in the bacterial coats of Bacillus and Clostridium organisms. These sasp gene targets may be identified by a technique described here as “heterologous PCR”. More specifically, these methods and materials provide means for specifically identifying certain members of the Bacillus genus, especially Bacillus anthracis and Bacillus globigii, and members of the Clostridium genus, especially Clostridium perfringens. Using heterologous PCR, novel gene targets are identified that yield species-specific regions for detection and identification. Based on this technique, assay techniques for identifying the organisms Bacillus anthracis, Bacillus globigii and Clostridium perfringens have been developed.

Another aspect of the invention comprises use of a small acid soluble protein (sasp-B) gene sequence in Bacillus anthracis that contains a specific region of stable nucleotide insertion absent in even the closest Bacillus relatives. This unique biomarker is a probe-binding region for the specific detection of Bacillus anthracis. Additional regions of sequence within the gene were used to develop a Bacillus anthracis specific amplification and detection system. The system is capable of distinguishing Bacillus anthracis from near neighbors during two phases of the amplified assay: during the amplification reaction, and by probe hybridization to the Bacillus anthracis specific biomarker within the amplified product for sequence confirmation.

As another aspect of the invention, the present Bacillus anthracis detection system either alone, or multiplexed with one or more additional genomic and/or virulence plasmid markers, is of use as a diagnostic or confirmatory tool in Public Health and clinical laboratories, as well as in the law enforcement sector. The system may be adapted to a variety of amplification and detection platforms in order to accommodate the technical and fiscal capabilities of the laboratory, or used in ‘field’ settings.

As yet another aspect of the invention, building upon the discovery that small acid-soluble spore protein genes maintain species-specific sequence signatures, analogous regions were identified among the sasp of Bacillus globigii (which is used as a non-pathogenic ‘surrogate’ for Bacillus anthracis in research and development applications, defense, and emergency response modeling, etc.). Taking advantage of this discovery, Bacillus globigii specific PCR was designed and reduced to practice.

Finally, using the same principals and strategy, primers were designed to a sasp gene sequence of Clostridium perfringens, (an acknowledged biological weapon) which was successfully amplified by the system.

Another aspect of the invention is to provide a two-step amplification/detection system. A particular sasp gene is selected for amplification, and its identity determined by a species-specific probe. Although the present invention is described in detail in connection with a PCR, it is understood that, based on the present teachings, other amplification and/or identification systems could be devised.

### BRIEF DESCRIPTION OF THE DRAWINGS

FIG. 1. is a multiple ClustalW DNA Sequence Alignment of sasp-B Amplicons from 38 Bacillus anthracis strains (Seq. ID No. 13 through 50). Bases 1-90 are in FIG. 1A, 91-180 are in FIGS. 1-B, and 181-240 are in 1C.

FIG. 2. is a ClustalW multiple sasp-B DNA Sequence Alignment of Bacillus anthracis, Bacillus thuringiensis and Bacillus cereus strains (Seq. ID No. 51 through 87). Bases 1-90 are in FIG. 2A, 91-180 are in FIGS. 2-B, and 181-240 are in 2C.

FIG. 3. is a representation of Bacillus globigii specific PCR primers targeting Bg sasp-gamma.

### DETAILED DESCRIPTION OF THE INVENTION

Described herein are regions of genomic sequence with patterns unique to the target organisms (Bacillus anthracis, Bacillus globigii and Clostridium perfringens) from which primers and probes were designed for specific amplification of target organism DNA, and where feasible, confirmation of amplicon sequence by probe hybridization. Spore coat and spore structural genes were studied because their products are intimately linked with the organism's environmental niche and, phenotype, and therefore distinct identity of each species. In the case of Bacillus anthracis, very little genomic sequence data is available, hence published sequence listings from closely related species were used as a starting point from which primers for amplification of Bacillus anthracis DNA (a process commonly known as ‘heterologous PCR’) were designed. Heterologous PCR, then, means PCR using primers known to hybridize to one target to amplify, under conditions of low stringency, another target, in this case, from another species of unknown sequence in the target gene. Some 27 spore genes were screened via heterologous PCR and scores of reaction products sequenced before a sufficiently definitive region of sequence was identified for anthracis specific primer and probe design. The signature which satisfied the specificity criteria, and which is the key to this invention, was found within the coding sequence of the sasp spore structural protein.

### EXAMPLE 1

### Database Search & Primer Design Example (Bacillus anthracis Primer/Probe Design)

Public databases GenBank and European Molecular Biology Laboratory (EMBL) were queried for “small acid-soluble spore protein” (sasp) DNA sequences. Three Bacillus cereus small acid-soluble protein genes were selected from GenBank for consideration (Bacillus cereus being one of the closest relatives to Bacillus anthracis). GenBank accession numbers for these sasp-B DNA sequences are: M13059 for Bacillus cereus sasp-1, M13060 for Bacillus cereus sasp-2, and M16813 for Bacillus cereus sasp-B.

Primer sequences were located within each sasp sequence which would maximize the likelihood of amplifying non-homologous sequences. For instance, whenever possible the 3′ end of a primer was concluded with one or more thymidine residues. Potential primer sequences were analyzed using Oligo 4.0 primer design software (National Biosciences, Plymouth, Minn.) for potential hairpin or concatomers, which might interfere with hybridization to target DNA. Also using Oligo 4.0 primer design software (National Biosciences, Plymouth, Minn.), primer sequences were adjusted to match their melting temperatures as closely as possible to one another, which generally enhances reaction specificity. The sequence similarity search tool BLAST was queried with the primer sequences in order to insure that the primers did not recognize any bacterial (or other microbial) sequences except the targeted Bacillus species. Primers were synthesized (Sequence IDs No. 1 through 6) using the PerSeptive Biosystems Expedite nucleic acid synthesis system (Perkin Elmer, Norwalk, Conn.). Oligos were released from columns by incubation in 29.3% ammonium hydroxide at 55° C. overnight, followed by evaporation of ammonium hydroxide using the SpeedVac 1SS110 (Savant Corp.). Primers were resuspended in 10 millimolar tris buffer, pH 8.3, and their concentration measured with a spectrophotometer.

Shown below are primers designed from Bacillus cereus sequences for heterologous PCR and sequencing of Bacillus anthracis, as described in Example 2.

B. cereus primers designed for heterologous PCR and sequencing of B. anthracis:

| Primer | | |
|---|---|---|
| name | Sequence (5′ to 3′) | |
| Bcsasp-B | 5′ ATGAGTAAAAAACAACAAGGTTAT | (SEQ ID NO: 1) |
| Bcsasp-B | 3′ CTGATTTGAGCTAGAAGATTGTGA | (SEQ ID NO: 2) |
| Bcsasp-1 | 5′ ATGGGAAAAAATAATAGTGGAAGT | (SEQ ID NO: 3) |
| Bcsasp-1 | 3′ GCGGTTAGCTCTACCACCAAGT | (SEQ ID NO: 4) |
| Bcsasp-2 | 5′ ATGTCAGCTAGCACAAATAAATT | (SEQ ID NO: 5) |
| Bcsasp-2 | 3′ TTATTTTTGGTAACCGCCTAA | (SEQ ID NO: 6) |

The primers are named according to their corresponding Bacillus cereus sasp.

### EXAMPLE 2

### Amplification of Bacillus Species Using Sasp Primers, and Analysis of Reaction Products

DNA was prepared according to the method described by Zhou et al. (Zhou, J., Bruns, M., and Jiedje, J. 1996. “DNA recovery from soils of diverse composition.” Appl. Environ. Microbiol. 62:316-322, 1996) from a non-infectious vegetative cells of Bacillus anthracis and Bacillus cereus type 168 (Bacillus Genetic Stock Center). The concentration of the purified DNA was determined by measuring the optical density at 260 nanometer wavelength using the Du 640 spectrophotometer (Beckman, Palo Alto), and by visual comparison to DNA standards of known quantity via agarose gel electrophoresis (Sambrook et al. Molecular Cloning, 1989, Cold Spring Harbor Laboratory Press).

For initial trial of the new primers, Tris buffer at pH 8.3, 8.8, and 9.2 was evaluated along with potassium chloride at (final) concentrations of 25 mM and 75 mM and magnesium chloride at (final) concentrations of 1.5 and 3.5 mM were screened using OptiPrime 10× buffers (Stratagene, La Jolla, Calif.), in order to determine favorable reaction conditions for the new primers. Reaction volumes were 100 microliters and contained approximately 100 nanograms of target DNA (and in the case of negative controls, no DNA).

A GeneAmp 9600 PCR System (Perkin Elmer, Norwalk, Conn.) was programmed as follows for thermalcycling of the reaction: A 5 minute 94 degree C. initial denaturation step was followed by 40 three step cycles of 94 degrees C. for 30 sec., 50 degrees C. for 30 sec., 72 degrees C. for 30 sec. A final extension step of 7 minutes at 72 degrees C. completed the thermalcycling.

Amplification products were viewed by ethidium bromide stained nusieve/agarose slab gel electrophoresis. Ten microliters of PCR product was mixed with 2 microliters of 6× gel loading dye (a filter sterilized solution of 0.25% bromophenol blue plus 40% (wt/vol) sucrose in double distilled sterile water). A molecular weight marker ladder of 100 base pair DNA fragments (Life Technologies, Gaithersburg, Md.) was run alongside the amplicons in order to gauge the size of the PCR products. The expected size of reaction products based on the primer locations within the B. cereus DNA sequence are: 213 base pairs for sasp-1, 198 base pairs for sasp-2, and 279 base pairs for sasp-B.

Results of this initial experiment are as follows: The major products (as judged by band intensity when viewed by gel) from amplification of Bacillus anthracis DNA were the same size as the major products of Bacillus cereus amplification using each of the three primers, regardless of the reaction mixture employed. Thus, it appeared that a Bacillus anthracis sasp gene sequence had been obtained.

### EXAMPLE 3

### DNA Sequence Analysis of Bacillus cereus Sasp-B Primed PCR Products from Bacillus anthracis

Bacillus anthracis PCR product from Example 2 was separated from primers and other reactants using the QIAquick Gel Extraction kit (Qiagen, Valencia, Calif.), and sequenced using the Dye Terminator Cycle Sequencing Kit (PE Biosystems, Foster City, Calif.) using the same Bacillus cereus sasp-B primers used to generate the product being sequenced. Sequencing reactions were performed in a TC-9600 thermalcycler (Perkin Elmer, Norwalk, Conn.). The completed sequencing reaction was electrophoresed and the sequence recorded by the Applied Biosystems Prism 377 Automated Sequencer (PE Biosystems, Foster City, Calif.). Since the PCR products were less than 300 bases, the entire length of each product was sequenced in a single run. The small size also made it possible to check the sequence by alignment of the two strands to one another. After inverting one strand with Gene Jockey sequence conversion software (BioSoft, Cambridge, UK), the strands were aligned using the Baylor College of Medicine Clustal W online multiple sequence alignment utility. CLUSTAL W is described in Thompson, J. D. Higgins, D. G. Gibson, T. J. (1994) as improving the sensitivity of progressive multiple sequence alignment through sequence weighting, position specific gap penalties and weight matrix choice. Nucl. Acids Res. 22:4673-4680. Where there was discrepancy between the two strands (i.e. where the bases did not match in the alignment), electrophoretograms of the sequencing gel run were examined (using the PE Biosystems Editview utility) in order to resolve the discrepancy.

Finished Bacillus anthracis sequence was aligned with Bacillus cereus published sequence and the differences analyzed. Of greatest interest was the six base region in the Bacillus anthracis sasp-B amplicon which was not present in the Bacillus cereus published sequence. Alignments of the Bacillus anthracis sequence with the published Bacillus cereus sequence for the region between each primer pair follow.

In these alignments, dots signify a match with the sequence shown; only mismatches are spelled out, in order to emphasize them. Primer sequences are not included, but would be extensions of the 5′ and 3′ ends of the sequences shown.

The Bacillus anthracis and B. cereus sasp-1 sequence alignment did not show significant differences

| | | | |
|---|---|---|---|
| B.cer | 1 | CGTAATGAAGTATTAGTTCGAGGCGCTGAACAAGCTCTTGATCAAATGAAATATGAAATT | (SEQ ID NO: 7) |
| B.anth | 1 | .........T.............T.................................... | (SEQ ID NO: 8) |
| B.cer | 61 | GCACAAGAGTTTGGTGTACAACTTGGTGCAGATACAACAGCTCGTTCAAACGGATCTGTT | |
| B.anth | 61 | ..................................................T......... | |
| B.cer | 121 | GGTGGTGAAATTACAAAACGTTTAGTAGCAATGGCAGAACA | |
| B.anth | 121 | ...................................T..... | |

The Bacillus anthracis and B. cereus sasp-2 sequence alignment did not show significant differences

| | | | |
|---|---|---|---|
| B.cer | 1 | AGCGGTTCCTGGTGCTGAATCAGCATTAGACCAAATGAAATACGAAATCGCTCAAGAGTT | (SEQ ID NO: 9) |
| B.anth | 1 | ............................................................ | (SEQ ID NO: 10) |
| B.cer | 61 | TGGTGTTCAACTTGGAGCTGATGCAACAGCTCGCGCTAACGGTTCTGTTGGTGGCGAAAT | |
| B.anth | 61 | ............................................................ | |
| B.cer | 121 | CACTAAACGTCTAGTTTCACTAGCTGAGCAACAA | |
| B.anth | 121 | .................................. | |

The Bacillus anthracis and B. cereus sasp-B sequence alignment showed a significant difference, namely a TAGCATT insert

| | | | |
|---|---|---|---|
| BcerPub | 1 | AACAAAGCAACTTCTGGTGCTAGCATTCAAAGTACAAATGCTAGTTATGGTACAGAGTTT | (SEQ ID NO: 11) |
| Banth | 1 | .....G..........................C........................... | (SEQ ID NO: 12) |
| BcerPub | 61 | TCAACTGAAACAGATGTACAAGCTGTAAAACAAGCAAACGCACAATCAGAAGCAAAGAAA | |
| Banth | 61 | G.G.........A..........A.............................T...... | |
| BcerPub | 121 | GCACAAGCTTCTGGTGCA------CAAAGTGCAAACGCTAGTTATGGTACAGAATTTGCA | |
| Banth | 121 | ..G..............TAGCATT.....CA....T........................ | |
| BcerPub | 175 | ACTGAAACAGACGTGCATTCTGTGAAAAAACAAAATGCTAAGTCAGCTGCAAAACAA | |
| Banth | 181 | ..................G...................AC.A............... | |

Conclusion from above results are as follows: Only the sasp-B sequence from Bacillus anthracis diverges from that of its near neighbor, B. cereus, to any useful extent. The sasp-2 sequences are identical, and the sasp-1 sequences differ too little to be of use in distinguishing the two organisms.

The underlined sequence TAGCATT (SEQ ID NO 107) represents an insertion region useful for distinguishing Bacillus anthracis from other Bacillus species.

### EXAMPLE 4

### Determination of Optimal Reaction Conditions for Maximal Sensitivity

By repeating the reaction condition optimization procedure described in Example 2 above, but upon a dilution series of Bacillus anthracis DNA down to 10 picograms per reaction, conditions were identified which resulted in maximum reaction sensitivity and specificity (as judged by the appearance of reaction products in ethidium bromide stained gel electrophoresis). By the criteria described, the best combination of conditions for use of BcSasp-B primers were as follows:

In a 100 microliter reaction volume the final concentration of reactants were 10 millimolar tris buffer pH 8.3; 25 millimolar potassium chloride; 2 millimolar magnesium chloride; 0.2 millimolar each dinucleotide triphosphate dATP, dCTP, dGTP, and dTTP; 50 picomoles of each primer, 5 units of Taq polymerase (Perkin Elmer, Norwalk, Conn.). Thermalcycling conditions were the same as those described in Example 2 above.

By viewing products with ethidium bromide stained gel electrophoresis it was determined that amplification specificity using these primers was quite good (only the expected product band and primers were visible), and product was visible down to 100 picograms per reaction.

### EXAMPLE 5

### Addressing the Necessary Question: How Conserved is the Sasp-B Sequence in Bacillus anthracis?

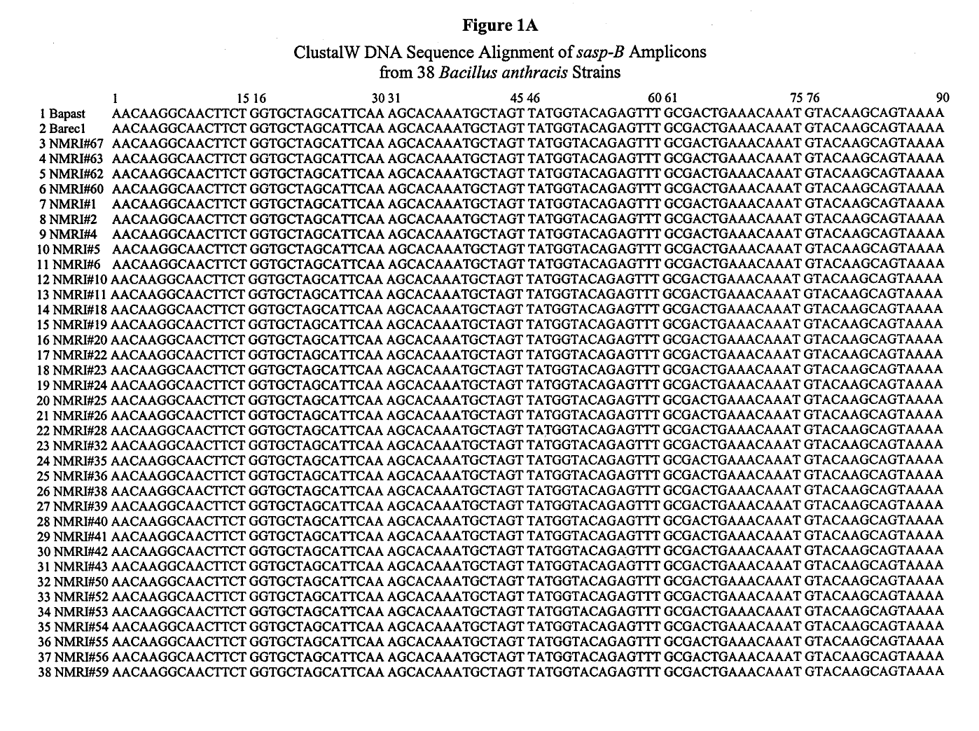

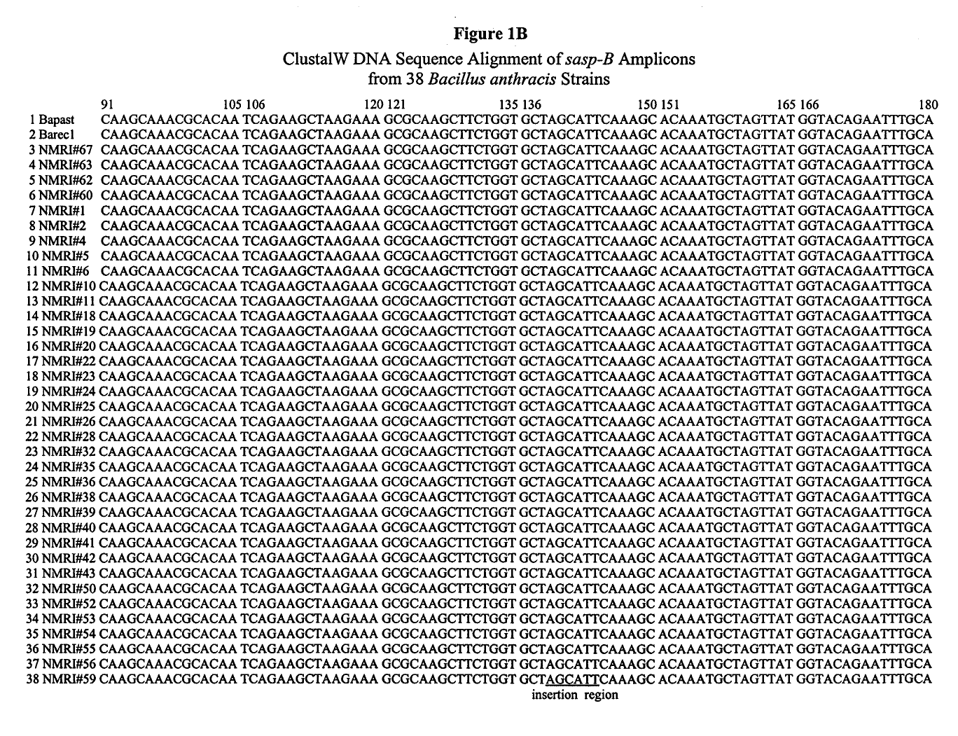

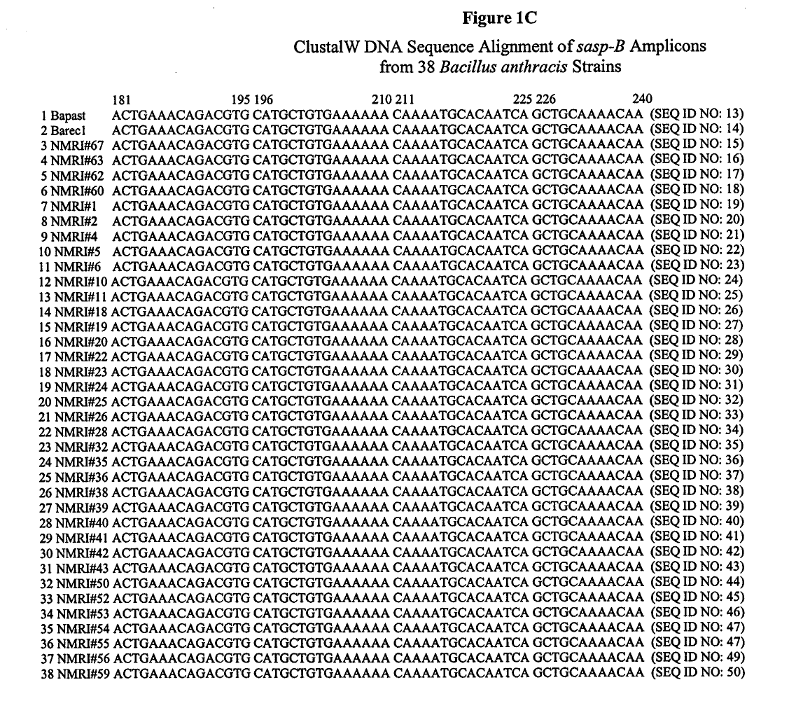

In order to establish how conserved the promising stretch of Bacillus anthracis sasp-B gene sequence is, DNA from a variety of B. anthracis isolates was amplified and the amplicons sequenced. We acquired DNA from 38 geographically diverse anthrax isolates which was prepared by staff of the Centre for Applied Microbiological Research, Porton Down, UK from the collection of Dr. Peter Turnbull. DNA from the 38 isolates was amplified using the B. cereus sasp-B primers and the (optimized) reaction conditions described in Example 4. The resulting PCR product was sequenced and analyzed as described above. The results presented in FIG. 1 confirmed that not only is the six base insertion present in diverse isolates of Bacillus anthracis, but the sequence as a whole is quite well conserved.

Referring now to FIGS. 1A-C, there is illustrated the results of the CLUSTAL W alignment of 38 different B. anthracis strains, with the sequence of interest underlined in line 38. It is conserved in all strains. The table below sets forth the identification of the various strains used:

TABLE 1

| Legend | | | | |
|---|---|---|---|---|
| Bacteria | ASC # | NMRI # | Designation | Description or ATCC # |
| Bacillus anthracis | | | Bapast | Institute Pasteur strain |
| Bacillus anthracis | | | Barec1 | UM23, Thorne strain |
| Bacillus anthracis | 152 | 1 | NMRI #1 | Namibia, 88 |
| Bacillus anthracis | BA40D 2 | | NMRI #2 | |
| Bacillus anthracis | 92 | 4 | NMRI #4 | Zambia, hippo, and passaged |
| | | | | mouse, 88 |
| Bacillus anthracis | 30 | 5 | NMRI #5 | Shropshire, acquired, 79 |
| Bacillus anthracis | 273 | 6 | NMRI #6 | XingJiang Province China, 92 |
| Bacillus anthracis | 63 | 10 | NMRI #10 | Etosha, soil via guinea pig, 86 |
| Bacillus anthracis | BA42D 11 | | NMRI #11 | |
| Bacillus anthracis | 237 | 18 | NMRI #18 | Landkey, soil, mouse |
| | | | | passage, 92 |
| Bacillus anthracis | 56 | 19 | NMRI #19 | Zimbabwe, human, 80 |
| Bacillus anthracis | 93 | 20 | NMRI #20 | Zambia, hippo, and passage |
| | | | | guinea pig, 88 |
| Bacillus anthracis | | 22 | NMRI #22 | MS191 |
| Bacillus anthracis | 91 | 23 | NMRI #23 | Zambia, hippo, 88 |
| Bacillus anthracis | 11 | 24 | NMRI #24 | NCTC5444, London, 28 |
| Bacillus anthracis | 64 | 25 | NMRI #25 | Russian vaccine, Sterne |
| Bacillus anthracis | 29 | 26 | NMRI #26 | Waybridge, cow, traced to |
| | | | | Senegal |
| Bacillus anthracis | 238 | 28 | NMRI #28 | Landkey, soil, 92 |
| Bacillus anthracis | 245 | 32 | NMRI #32 | Sterne |
| Bacillus anthracis | | 35 | NMRI #35 | 1 + 2 + DMD |
| Bacillus anthracis | 234 | 36 | NMRI #36 | Wessex, soil, 92 |
| Bacillus anthracis | | 38 | NMRI #38 | Rvacc |
| Bacillus anthracis | | 39 | NMRI #39 | PR13P |
| Bacillus anthracis | 264 | 40 | NMRI #40 | Zambia, contaminated soil, 91 |
| Bacillus anthracis | 43 | 41 | NMRI #41 | M36, passaged rabbits and rats |
| Bacillus anthracis | 69 | 42 | NMRI #42 | New Hampshire, 57 |
| Bacillus anthracis | 65 | 43 | NMRI #43 | Brazil, cow, acquired, 82 |
| Bacillus anthracis | 192 | 50 | NMRI #50 | Landkey, soil |
| Bacillus anthracis | 4 | 52 | NMRI #52 | M36, passaged in rabbits |
| Bacillus anthracis | 3 | 53 | NMRI #53 | M36, derived from original |
| | | | | challenge stock |
| Bacillus anthracis | 2 | 54 | NMRI #54 | Griunard, 1950s |
| Bacillus anthracis | 54 | 55 | NMRI #55 | Zimbabwe, human, 80 |
| Bacillus anthracis | | 56 | NMRI #56 | 1 + 2 − |
| Bacillus anthracis | 28 | 59 | NMRI #59 | Waybridge, cow, traced to |
| | | | | Senegal, 78 |

NMRI is the Naval Medical Research Institute, Bethesda, Maryland
ATCC is the American Type Culture Collection
ASC is The Association Of Systematics Collections

### EXAMPLE 6

### Scrutinizing the Sequence of BcSasp-B Primed Amplicons from Bacillus anthracis Near Neighbors

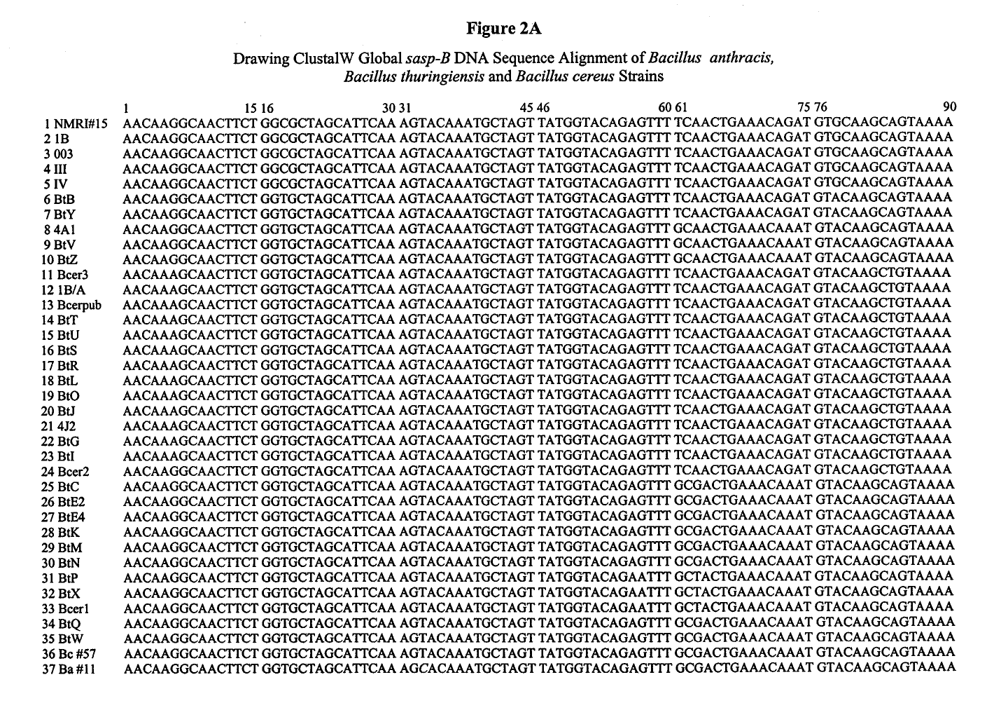

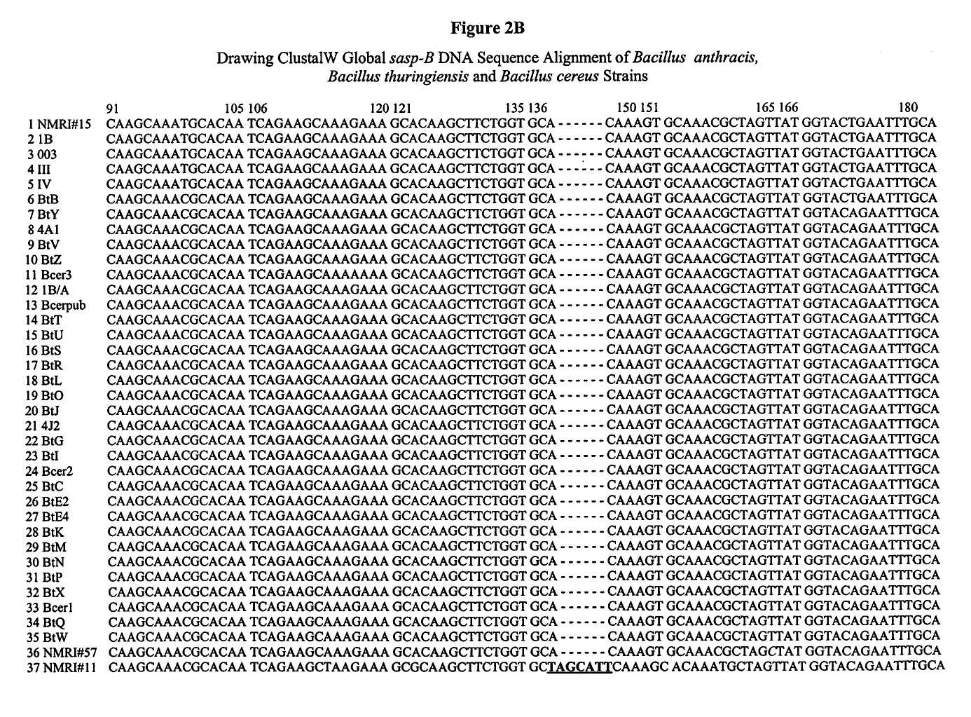

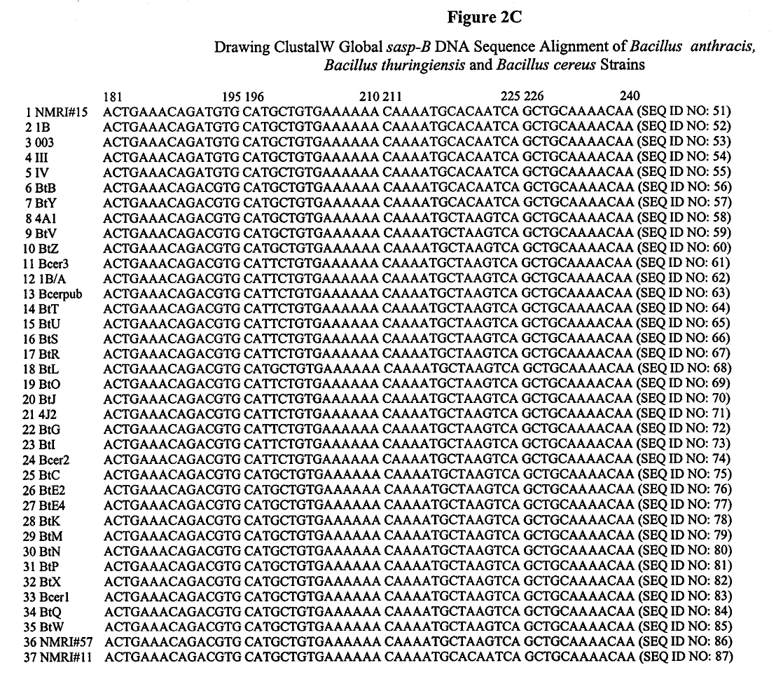

Following reports that other labs had mistakenly identified B. thuringiensis as Bacillus anthracis, 24 serotypes of B. thuringiensis were obtained in order to check whether the sequence of sasp-B amplicons resembled those of other amplicons. DNA was prepared from B. thuringiensis as well as B. cereus liquid cultures following the method described by Zhou (Zhou, J., Bruns, M., and Jiedje, J. 1996. “DNA recovery from soils of diverse composition.” Appl. Environ. Microbiol. 62:316-322), and amplified 100 nanograms of the DNA using B. cereus sasp-B primers. Product fragments were gel purified, extracted, sequenced, and analyzed as described in Example 3 above. The resulting alignment (FIG. 2) includes B. thuringiensis and B. cereus sasp-B sequences as well as Bacillus anthracis sasp-B sequence from the previous example:

Referring now to FIGS. 2A, 2B, and 2C, the single Bacillus anthracis sequence (#37 which is the bottom row of FIGS. 2A, 2B, & 2C) shows a unique pattern of sequence divergence from the sasp-B sequence of these near neighbor isolates. The identities of the sequences are shown in Table 2 below:

TABLE 2

| Legend: | | | | |
|---|---|---|---|---|
| Bacteria | BGSC # | Serotype | | Designation |
| Bacillus lishenformis, | | 5A2 | | 5A2 |
| Bacillus thuringiensis | | 4A1 | serot-1 | 4A1 |
| Bacillus thuringiensis | | 4A3 | cry (thur) serot-1 | 4A3 |
| Bacillus thuringiensis | | 4J2 | aizawai, pacificus/serot-7 | 4J2 |
| Bacillus thuringiensis | HD3 | 4B2 | 2 standard | BtB |
| Bacillus thuringiensis | HD4 | 4C3 | 3a standard | BtC |
| Bacillus thuringiensis | HD7 | 4E2 | 4a4b dendrolimus standard | BtE2 |
| Bacillus thuringiensis | | 4E4 | 4a4b | BtE4 |
| Bacillus thuringiensis | HD29 | 4G5 | 5a5b | BtG |
| Bacillus thuringiensis | HD10 | 4I1 | 6 | BtI |
| Bacillus thuringiensis | HD11 | 4J4 | 7 | BtJ |
| Bacillus thuringiensis | HD12 | 4K1 | 8 standard | BtK |
| Bacillus thuringiensis | HD537 4L3 | 9 standard | | BtL |
| Bacillus thuringiensis | HD146 4M1 | 10 standard | | BtM |
| Bacillus thuringiensis | HD201 4N1 | 11 antisera standard | | BtM |
| Bacillus thuringiensis | HD542 4O1 | 12 standard | | BtO |
| Bacillus thuringiensis | HD395 | 4P1 | 13 standard | BtP |
| Bacillus thuringiensis | ONR60A | 4Q1 | 14 | BtQ |
| Bacillus thuringiensis | HD511 4R1 | 15 | | BtR |
| Bacillus thuringiensis | HD521 4S2 | 16 standard | | BtS |
| Bacillus thuringiensis | HD525 4T1 | no flagellar antigen | | BtT |
| Bacillus thuringiensis | HD541 4U1 | 11a11c | | BtU |
| Bacillus thuringiensis | | 4V1 | 17 | BtV |
| Bacillus thuringiensis | HD 867 | 4W1 | 18 | BtW |
| Bacillus thuringiensis | IS720 | 4X1 | 21 | BtX |
| Bacillus thuringiensis | HD868 | 4Y1 | 19 standard | BtY |
| Bacillus thuringiensis | HD501 | 4Z1 | 8a8c standard | BtZ |
| Bacillus anthracis | BA42D | 11 | | NMRI #11 |
| Unidentified Bacillus | | | | 003 |

| Unidentified Bacillus | | Taken from filled bag in “final mixing | 1B |
|---|---|---|---|
| | | trailer” | |
| Unidentified Bacillus | | Isolated from 1B culture as morphologically | 1B/A |
| | | distinct colonies | |
| Unidentified Bacillus | | Isolated from 25 kg media drum, bentonite | III |
| | | mixture | |
| Unidentified Bacillus | | Isolated from bentonite spore stock | IV |
| Bacillus cereus | Genbank | NCBI Genbank database | Bcerpub |
| | #M16813 | | |
| Bacillus cereus | ATCC | Purchased from ATCC | Bcer1 |
| | 14579 | | |
| Bacillus cereus | ATCC | Purchased from ATCC | Bcer2 |
| | 11778 | | |
| Bacillus cereus | ATCC 6464 | Purchased from ATCC | Bcer3 |

BGSC is the Bacillus Genetic Stock Center, at The Ohio State University

Based on the DNA sequence information in FIGS. 1 and 2, amino acid sequences were extrapolated and evaluated for the sasp-B genes from Bacillus anthracis, Bacillus cereus and Bacillus thuringiensis. These extrapolated sequences are shown below in an extrapolated amino acid sequence alignments for the sasp-B gene from Bacillus anthracis, Bacillus cereus and Bacillus thuringiensis. The identities of the sequences are own in Table 3.

| | | | |
|---|---|---|---|
| 1 | | 15 16 30 31 45 46 60 | |
| 1 | 4D4 | NKATSGASIQSTNAS YGTEFSTETDVQAVK QANAQSEAKKAQASG A-QSANASYGTEFA | (SEQ ID NO: 88) |
| 2 | Bcep | NKATSGASIQSTNAS YGTEFSTETDVQAVK QANAQSEAKKAQASG A--QSANASYGTEFA | (SEQ ID NO: 89) |
| 3 | BtK | NKATSGASIQSTNAS YGTEFATETNVQAVK QANAQSEAKKAQASG A--QSANASYGTEFA | (SEQ ID NO: 90) |
| 4 | BtB | NKATSGASIQSTNAS YGTEFSTETDVQAVK QANAQSEAKKAQASG A--QSANASYGTEFA | (SEQ ID NO: 91) |
| 5 | Banth | NKATSGASIQSTNAS YGTEFATETNVQAVK QANAQSEAKKAQASG ASIQSTNASYGTEFA | (SEQ ID NO: 92) |
| 6 | Bmyc | NKATSGASIQSTNAS YGTEFATETNVQAVK QANAQSEAQKAQASA A--QSANASYGTEFA | (SEQ ID NO: 93) |
| | | 61 75 76 | |
| 1 | 4D4 | TETDVHSVKKQNAKS AAKQ | |
| 2 | Bcep | TETDVHSVKKQNAKS AAKQ | |
| 3 | BtK | TETDVHAVKKQNAKS AAKQ | |
| 4 | BtB | TETDVHAVKKQNAQS AAKQ | |
| 5 | Banth | TETDVHAVKKQNAQS AAKQ | |
| 6 | Bmyc | TETDVHAVKKQNAQS AAK | |

TABLE 3

| Legend: | | | | |
|---|---|---|---|---|
| Bacteria | BGSC # | Serotype | | Designation |
| Bacillus thuringiensis | type strain | | | 4D4 |
| Bacillus thuringiensis | HD12 | 4K1 | 8 standard | BtK |
| Bacillus thuringiensis | HD3 | 4B2 | 2 standard | BtB |
| Bacillus cereus published sequence | | GenBank #M16813 | | Bcerp |
| Bacillus mycoides | | | | ATCC 6421, subtype Flugge |
| Bmyc | | | | |
| Bacillus anthracis | | | | Banth |

### EXAMPLE 7

### Design and Evaluation of Primers and Probes to Bacillus anthracis Specific Sasp-B DNA Sequence

In the previous examples, the BcSasp-B primers were useful for evaluating the prevalence of the unique Bacillus anthracis sasp-B signature, but sequencing was required to distinguish amplicons of the several Bacillus species which could be amplified using the Bacillus cereus primers. By studying the alignment of Bacillus anthracis and Bacillus cereus sasp-B sequences (above) potential anthracis specific primer and probe sites were identified (shown below, SEQ ID NO: 94 and 95). Eight oligonucleotides were designed with the aid of Oligo 4.0 and BLAST database search utilities then synthesized (all as described in Example 1 above) and evaluated experimentally in various combinations for their ability to prime amplification of Bacillus anthracis only, using a panel of near neighbor Bacillus species. Three of the primer pairs were designed to incorporate the Bacillus anthracis insertion region into the three prime end of one primer per pair. This strategy greatly limited amplicon size and did not leave any Bacillus anthracis specific sequence for probe design.

The combination of primers originally designated BaSPB7 and BaSPB8 (below) were sufficiently specific. From 100 nanograms B. cereus target a very faint product band of nearly (but not exactly) the correct size, was evident; when compared to signals from an amplified dilution series of Bacillus anthracis DNA, the signal from Bacillus cereus was approximately equivalent to product from 10 picograms—indicating 10,000 fold less efficient amplification. Bands were not visible at or near the correct size from products of Bacillus coagulans, Bacillus circulans, Bacillus globigii, Bacillus mycoides, Bacillus subtilis or Bacillus thuringiensis amplification.

In addition, these primers were for sequences flanking, rather than incorporating the Bacillus anthracis insertion region, thus leaving this region within the product for binding to probes designed to hybridize to this unique signature. The Bacillus anthracis primer data (from analysis by Oligoprimer design software, National Biosciences, Plymouth, Minn.) is summarized as follows:

BaSPB7 primer sequence:

5′ GTT ATG GTA CAG AGT TTG CG 3′ (SEQ ID NO: 94)

Tm=57.4° C. (salt 1000.0 mM; oligo 0.6 pM)

Td=57.6° C., G(25° C.)=−34.7 kcal/mol, Mr=6283

Ext. coeff.: 5.05 nmol/A260, 31.7 μg/A260

BaSPB8 primer sequence:

5′ TTG TTT TGC AGC TGA TTG T 3′ (SEQ ID NO: 95)

Tm=58.3 ° C. (salt 1000.0 mM; oligo 0.6 pM)

Td=58.9 ° C., G(25° C.)=−34.1 kcal/mol, Mr=5911

Ext. coeff.: 5.82 nmol/A260, 34.4 μg/A260

Optimal amplification conditions were determined in the same manner described in Examples 2 and 4 above. Optimal amplification conditions were identified as follows:

Thermalcycling: Amplifications were performed in a Perkin Elmer 9600 thermocycler with the following thermal cycling regime: 94° C. for 5 minutes, then 40 repeating cycles of 94° C. for 30 seconds, 50° C. for 30 seconds and 72° C. for 30 seconds, followed by a 7 minute 72° C. final extension step.

Reaction mixture: Each 100 μl reaction contained 0.1 millimolar each dATP, dCTP, dGTP and dTTP, 25 picomoles each primer, 10 millimolar Tris-HCl pH 8.3, 2 millimolar MgCl2, 25 millimolar KCl, 2.5 units of Taq polymerase (Perkin Elmers, Norwalk, Conn.) and 100 ng or less of template DNA.

The uniqueness of these primers may be seen by a Bacillus anthracis and Bacillus cereus sasp-B sequence alignment emphasizing Bacillus anthracis specific primer sequences:

| | | | |
|---|---|---|---|
| BcerPub | 1 | AACAAAGCAACTTCTGGTGCTAGCATTCAAAGTACAAATGCTAGTTATGGTACAGAGTTT | (SEQ ID NO: 96) |
| Banth | 1 | .....G..........................C..........GTTATGGTACAGAGTTT | (SEQ ID NO: 97) |
| | | --primer BaSPB7-- | |
| BcerPub | 61 | TCAACTGAAACAGATGTACAAGCTGTAAAACAAGCAAACGCACAATCAGAAGCAAAGAAA | |
| Banth | 61 | GCG.........A..........A.............................T...... | |
| BcerPub | 121 | GCACAAGCTTCTGGTGCA------CAAAGTGCAAACGCTAGTTATGGTACAGAATTTGCA | |
| Banth | 121 | ..G..............TAGCATT.....CA....T........................ | |
| BcerPub | 175 | ACTGAAACAGACGTGCATTCTGTGAAAAAACAAAATGCTAAGTCAGCTGCAAAACAAA | |
| Banth | 181 | ..................G...................ACAATCAGCTGCAAAACAAA | |
| | | <--primer BaSPB8- | |

The Bacillus anthracis and Bacillus ceurus sasp-B sequence alignment shows the sequence similarity between the present Bacillus anthracis sasp B DNA (which is a 240 base pair amplicon as described above) and the corresponding region of its most similar known sequence, the Bacillus ceurus sasp B gene. This alignment, run in the CLUSTALW program described herein, yields a similarity score of 89%, using default parameters. As is known in the art, the default parameters for nucleic acid pairwise alignments are gap opening penalty=15; gap extension penalty=6.66. The IUB matching protocol is used—All matches score 1.9; all mismatches for IUB symbols score 0. The CLUSTAL matching protocol can also be used. In this case, matches score 1.0 and mismatches score 0.0. In either case, CLUSTALW comparison of the Bacillus anthracis sasp B DNA and the comparable Bacillus ceurus sasp B DNA yields a score of 89%.

Similarly, given the present Bacillus anthracis sasp B DNA, a similarity score from the NCBI GenBank nucleotide database using BLAST (default parameters, version 2.2.1) is easily obtained. As is known in the art, the BLAST defaults for nucleotide sequences are: −3 for a nucleotide mismatch; +1 for a nucleotide match; and 0 gap penalty. The matrix used by default is Blosum62.

In the present case, a BLAST search revealed that the closest sequence corresponds to the Bacillus ceurus small acid-soluble spore protein, Accession Number M16813. This corresponds with the discussion in this Example. Using BLAST parameters, the two nucleotide sequences have an identity of 86%.

A manual analysis of the Bacillus anthracis and Bacillus cereus sasp-B-sequence alignment yields a similarity score of 90-91%. To perform a manual analysis, one of skill in the art would add the number of nucleotides that differ among the two sequences. One of skill would then subtract that number from the total number of nucleotides in the sasp-B Bacillus anthracis sequence and divide the resulting number by the total number of nucleotides in the sasp-B Bacillus anthracis sequence. The resulting number multiplied by 100 yields the similarity score.

Accordingly, those of skill in the art would recognize that the Sasp-B DNA of Bacillus anthracis is homologous but not identical to that of Bacillus cereus. Thus, this invention includes any sasp-B from Bacillus anthracis that might include minor single base differences (polymorphisms) from SEQ ID NO: 97 (or identical SEQ ID NO: 87), yet, maintain the insert of SEQ ID NO: 107.

### EXAMPLE 8

### Selection and Evaluation of Probes for Detection of Bacillus anthracis Sasp-B Amplicons

Three of the oligonucleotides evaluated as primers incorporated the Bacillus anthracis specific insertion region, and having designed primers flanking the insertion region, these oligos were tested as probes to confirm the identity of the amplicons; only amplicons from B. anthracis would include the 6 base insertion, as follows:

Alignment of Bacillus cereus and Bacillus anthracis Sasp-B sequences emphasizing probed locations

| | | |
|---|---|---|
| Bcer | AACAAAGCAACTTCTGGTGCTAGCATTCAAAGTACAAATGC | (SEQ ID NO: 98) |
| Banth | AACAAGGCAACTTCTGGTGCTAGCATTCAAAGCACAAATGC | (SEQ ID NO: 99) |
| Bcer | TAGTTATGGTACAGAGTTTTCAACTGAAACAGATGTACAAGCTGTAAAACAAGCAAACGCACAA | |
| Banth | TAGTTATGGTACAGAGTTTGCGACTGAAACAAATGTACAAGCAGTAAAAAAGCAAACGCACAAT | |
| Bcer | TCAGAAGCAAAGAAAGCACAAGCTTCTGGTGCA------AAAGTGCAAACGCTAGTTATGGTACAGAATTTGCAA | |
| Banth | CAGAAGCTAAGAAAGCGCAAGCTTCTGGTGCTAGCATTCAAAGCACAAATGCTTTGCATAGTTATGGTACAGAAA | |
| | ------------------- | |
| | --------------------- | |
| | ↑location of probes tested ↓ | |
| Bcer | CTGAAACAGACGTGCATTCTGTGAAAAAACAAAATGCTAAGTCAGCTGCAAAACAA | |
| Banth | CTGAAACAGACGTGCATGCTGTGAAAAAACAAAATGCACAATCAGCTGCAAAACAA | |

Three oligonucleotides evaluated for use as Bacillus anthracis probes:

| | | |
|---|---|---|
| BaSPB2: (inverted, ‘lower strand’ sequence): | 5′ GCATTTGTGCTTTGAATGCTA 3′ | (SEQ ID NO: 100) |
| BaSPB4: (inverted, ‘lower strand’ sequence): | 5′ CATTTGTGCTTTGAATGCTA 3′ | (SEQ ID NO: 101) |
| BaSPB5: (direct, ‘upper strand’ sequence): | 5′ AGCTTCTGGTGCTAGCATT 3′ | (SEQ ID NO: 102) |

Oligos (shown above) were tested as probes in the following manner:

Bacillus anthracis DNA (100 nanograms per 100 microliter reaction of the Sterne strain) was amplified using Biotin labeled BaSPB7 and BaSPB8 primers which had been synthesized to our order by Life Technologies (Gaithersburg, Md.).

Following the instructions for the Universal GeneComb Kit (Bio-Rad, Richmond, Calif.), 8 picomoles and 5 picomole quantities of each oligo (henceforth called probe) in the kit binding buffer was spotted on the GeneComb test kit card nitrocellulose surface, and bound to the nitrocellulose using the UV Stratalinker 1800 (Stratagene, La Jolla, Calif.) set to auto crosslink mode. The biotinylated amplicon was denatured, and allowed to migrate across the (bound) probe spots, hybridizing to them in the process. The resulting bound biotinylated hybrid was then reacted with the kit streptavidin/alkaline phosphatase conjugate followed by kit chromogenic substrate-enabling visualization of the probe/amplicon hybrids. Evaluation of the test spot signal intensity guided the choice of probe for further evaluation. BaSPB4 proved most sensitive in this test, so succeeding work was done with this probe.

In order to roughly gauge sensitivity of the system, amplification was performed using Biotinylated BaSPB7 and BaSPB8 primers, the target(s) being a dilution series of Bacillus anthracis DNA (Sterne strain) whose concentration (prior to dilution) had been carefully determined using the Beckman DU 640 Spectrophotometer. The dilutions amplified were (total input per 100 microliter reaction): 10, 5, and 1 picogram(s), 100, 50, and 20 femtograms. Following the GeneComb test kit instructions, one tenth of each PCR was reacted with (bound) BaSPB4 and the resulting hybrids ‘developed’. The developed test strips reflected the input DNA dilutions in color intensity, with even the 20 femtogram reaction yielding a visible spot. (Data not shown.) The results comprised a visible spot for each dilution tested (10 pg down to 20 fg), with the negative PCR and kit control showing no spots.

Finally, probe BaSPB4 was tested for specificity. Fifty nanograms of DNA from each of the following Bacillus species was amplified using biotinylated BaSPB7 and BaSPB8: Bacillus anthracis, Bacillus thuringiensis, Bacillus cereus, Bacillus mycoides, B. subtilis, Bacillus globigii. The denatured amplicon of each DNA species was reacted against the bound BaSPB4 probe and the test strips developed. Only the Bacillus anthracis amplicon resulted in any signal (which was quite intense); none of the other species bound to the probe in order to result in a signal. Conclusion: BaSPB4 binds Bacillus anthracis DNA specifically. BaSPB4 binding specificity was demonstrated with the BioRad universal GeneComb System (data not shown). 100 ng of each species of DNA amplified was placed on each panel, as follows (1-8):

Species Amplified:

1) Bacillus anthracis

2) Bacillus thuringiensis

3) Bacillus cereus

4) Bacillus mycoides

5) Bacillus subtilis

6) Bacillus globigii

7) Negative PCR

8) Kit positive control

The Bacillus anthracis showed a large spot; the other panels were blank, except for the positive control. This showed that only the amplified Bacillus anthracis sasp B sequence reacted with the probe.

Some potential detection systems for the Bacillus anthracis specific primer/probe system are as follows:

The primer/probe system described above is ideally suited to the 5′nuclease fluorescence homogeneous assay in which the accumulation of specific amplicon is monitored as fluorescence is released from the probe by Taq polymerase during the amplification of target DNA to which the probe anneals. This system is described in U.S. Pat. Nos. 5,538,848; 5,723,591; and 5, 876,930, hereby incorporated by reference.

The primer/probe system described is also suited to amplification followed by separate hybridization of biotinylated amplicon molecules to bound probe in a colormetric microwell plate type assay (J. Mulder, N. McKinney, C. Christopherson, J. Sninsky, L. Greenfield and S. Kwok. Rapid and Simple PCR Assay for Quantitation of HIV-I RNA in Plasma: Applications to Acute Retroviral Infection. J. Clin. Micro. 37(2): 292-300, 1994); or, in similar manner, as with the automated AmpliCore integrated PCR+detection devices (Roche, Pleasanton, Calif.), and as described, a simple dot blot type assay.

There is an increasing number of PCR devices and coordinated detection strategies; the primers/probes and amplification system described are robust, specific, and sensitive enough to be adapted to most of these.

Regardless of detection format, the described assay could be used to monitor the presence of anthrax in the environment (such as for investigation by military and law enforcement agencies of clandestine production of anthrax for illicit use; for Public Health and law enforcement agencies to test suspicious spore-like powders; to check for anthrax in suspected cases of bio-terrorist attack).

This assay is also well suited as a rapid, specific, and sensitive method for detecting anthrax in biological fluids such as blood, sputum, and feces in clinical and Public Health labs, as well in the field, and for autopsies.

### EXAMPLE 9

### Development of Bacillus globigii Specific PCR Based on Sasp Gene Sequence

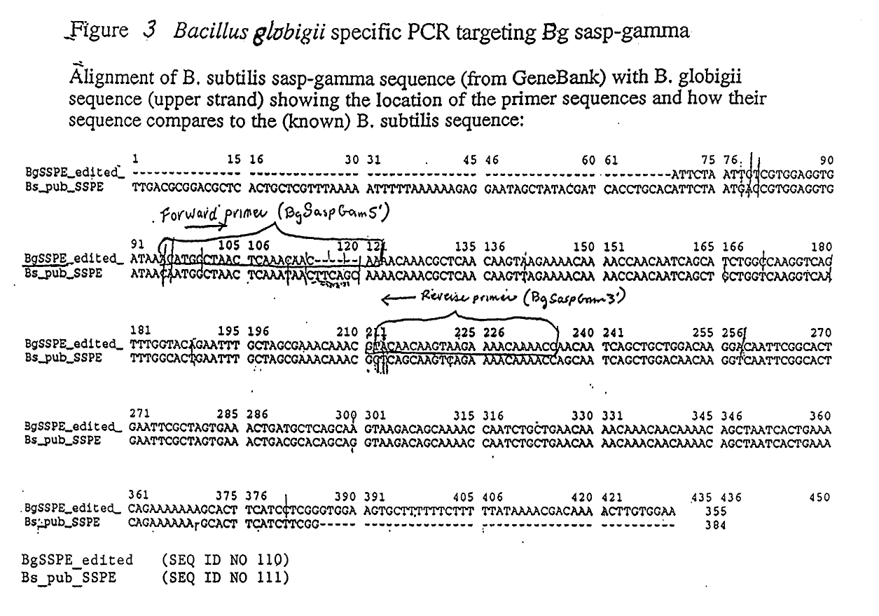

In the same manner as described in Example 1 above, a sasp gene (sasp-gamma in this case) sequence was identified for the production of primers specific for Bacillus globigii sequence. Primers and amplification conditions were designed (see FIG. 3) for heterologous PCR based on published sequence for the Bacillus subtilis sasp E gene (sasp-gamma) acquired from GenBank (accession number M16184). After sequencing amplicons from Bacillus globigii (generated using the Bacillus subtilis primers), and aligning Bacillus globigii sequence with the published Bacillus subtilis sequence, Bacillus globigii specific primers were designed taking advantage of the differences in the sequence. After searching the databases to be sure that the new Bacillus globigii primers were not homologous to other sequences, and optimizing amplification conditions, a panel of Bacillus species were amplified to check primer specificity. Amplicons of the correct size were produced only from Bacillus designated as Bacillus globigii, for all but the most arcane intents and purposes (there is disagreement among a very few researcher as to whether Bacillus subtilis niger and Bacillus atrophaeus are, in fact, genetically different from Bacillus globigii at all); importantly, the new primers did not amplify Bacillus subtilis or Bacillus amyloliquifaciens—which are distinct species, yet very closely related to Bacillus globigii. Referring now to FIG. 3, there is shown an alignment of Bacillus subtilis sasp-gamma sequence (from Genbank) (Bs_pub_SSPE) with Bacillus globigii sequence (upper strand) showing the location of the primer sequences and how their sequence compares to the known Bacillus subtilis sequence.

The BgSaspGam primers produce B globigii specific PCR product, as was demonstrated in an Nuseive-Agarose gel (data not shown). The gel showed approximately a 135b Bacillus globigii specific amplicon. No amplification of negative controls in Bacillus cereus; Bacillus amyoliquifaciens, Bacillus megaterium, or Bacillus globisporus was observed. Amplification was observed with Bacillus atropheus (ATCC 6455 and 49337) and Bacillus niger. It should be noted that Bacillus subtilis niger and Bacillus atrophaeus have been officially designated Bacillus globigii since they are virtually indistinguishable from Bacillus globigii at the molecular level. Near neighbors Bacillus subtilis, Bacillus globisporous and Bacillus megatarium do not amplify with the BgSaspGam primers.

Bacillus globigii sasp-gamma primers:

| | |
|---|---|
| BgSaspGam 5′ | |
| 5′ ACATGGCTAACTCAAACAACAA 3′ | (SEQ ID NO: 103) |
| BgSaspGam 3′ | |
| 5′ GGTTTTGTTTTCTTACTTGTTGTAC 3′ | (SEQ ID NO: 104) |

Reaction conditions successfully employed using the above primers are as follows:

Reaction mixture composition: In a 100 microliter reaction volume the final concentration of reactants were 10 millimolar tris buffer pH 8.3; 25 millimolar potassium chloride; 2 millimolar magnesium chloride; 0.2 millimolar each dinucleotide triphosphate dATP, dCTP, dGTP, and dTTP; 50 picomoles of each primer, 5 units of Taq polymerase (Perkin Elmer, Norwalk, Conn.).

Thermalcycling conditions using TC9600 (Perkin Elmer, Norwalk, Conn.): A 5 minute 94 degree C. initial denaturation step was followed by 40 three step cycles of 94 degrees C. for 30 sec., 50 degrees C. for 30 sec., 72 degrees C. for 30 sec. A final extension step of 7 minutes at 72 degrees C. completed the thermalcycling.

There a number of potential uses of the research Bacillus globigii specific amplification system. Bacillus globigii is used as a nonpathogenic surrogate, replacing Bacillus anthracis, for purposes of modeling aerosol spore distribution under various environmental conditions, as well as for testing spore collection hardware. The agencies carrying out these endeavors have had trouble finding a way of detecting only the Bacillus globigii used in their experiments; detection systems have been non-specific, resulting in false alarms and compromised data.

### EXAMPLE 10

### Development of Clostridium perfringens PCR Based on Sasp Gene Sequence

In a manner similar to the above descriptions, a sasp gene (sasp-2 in this case) sequence was identified for the production of primers for amplification of Clostridium perfringens sequence. Primers and amplification conditions were designed and carried out using Clostridium perfringens DNA. While amplification successfully produced product of the correct size (when viewed by ethidium bromide gel electrophoresis), near neighbor DNA has yet to be evaluated in order to assess specificity of these primers.

Clostridium perfringens sasp-2 primers:

| | |
|---|---|
| CPssp2-1: | |
| 5′ AATAACTAAGGAGGAATGAAAAATGT 3′ | (SEQ ID NO: 105) |
| Cpssp2-2: | |
| 5′ TTGTTCTACCATTCTTTTAACCATT 3′ | (SEQ ID NO: 106) |

The following reaction conditions were successfully employed using the above primers:

Reaction mixture composition: In a 100 microliter reaction volume the final concentration of reactants were 10 millimolar tris buffer pH 8.3; 25 millimolar potassium chloride; 2 millimolar magnesium chloride; 0.2 millimolar EACH dinucleotide triphosphate dATP, dCTP, dGTP, and dTTP; 50 picomoles of each primer, 5 units of Taq polymerase (Perkin Elmer, Norwalk, Conn.).

Thermalcycling conditions using TC9600 (Perkin Elmer, Norwalk, Conn.): A 5 minute 94 degree C. initial denaturation step was followed by 40 three step cycles of 94 degrees C. for 30 sec., 50 degrees C. for 30 sec., 72 degrees C. for 30 sec. A final extension step of 7 minutes at 72 degrees C. completed the thermalcycling.

There are a number of potential uses of Clostridium perfringens specific PCR. Clostridium perfringens is officially listed as a biological weapon agent, so uses would be similar to those described for the Bacillus anthracis specific primers.

Also, using these primers for heterologous PCR of Clostridium botulinum (a serious health threat and potential biological weapon) in order to acquire sequence information for the design of primers specific to that organism. Such primers, and any probe so identified in the process, would also be useful in the same manner described for the Bacillus anthracis specific detection research system described above.

Having described the present invention, it will be apparent that other embodiments are possible in light of the present teachings. For example, other DNA amplification methods besides PCR are known, such as the Q-beta replicase method. Certain of these methods may be used in a single-step amplification/detection protocol, based, for example, on the unique Bacillus anthracis sasp-B insertion TAGCATT (SEQ ID NO 107).

Accordingly, the present invention should be understood to encompass subject matter limited only by the scope of the appended claims. Furthermore, having described the amino acid structure of at least a portion of the B. anthracis sasp-B gene, tests which directly test the sasp-B protein are now possible.

### EXAMPLE 11

### The Production of Antibodies to Sasp Polypeptides

The novel sasp polypeptides of the invention can also be used to produce antibodies which are specifically immunoreactive or bind to epitopes of the sasp polypeptides. Antibodies of the invention specifically include antibodies which bind to unique polypeptides produced by the B. anthracis DNA sequence shown in Example 6 (and translated in Example #5) and identified as NMRI#11.

The term “antibody” as used in this invention includes intact molecules as well as fragments thereof which are capable of binding the epitopic determinant. These antibody fragments retain some ability to selectively bind with its antigen or receptor and are defined as follows: Fab, the fragment which contains a monovalent antigen-binding fragment of an antibody molecule, can be produced by digestion of whole antibody with the enzyme papain to yield an intact light chain and a portion of one heavy chain; Fab′, the fragment of an antibody molecule can be obtained by treating whole antibody with pepsin, followed by reduction, to yield an intact light chain and a portion of the heavy chain; two Fab′ fragments are obtained per antibody molecule; F(ab′).sub.2, the fragment of the antibody that can be obtained by treating whole antibody with the enzyme pepsin without subsequent reduction; F(ab′).sub.2 is a dimer of two Fab′ fragments held together by two disulfide bonds; Fv, defined as a genetically engineered fragment containing the variable region of the light chain and the variable region of the heavy chain expressed as two chains; and single chain antibody (“SCA”), defined as a genetically engineered molecule containing the variable region of the light chain, the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York (1988), incorporated herein by reference).

As used in this invention, the term “epitope” means any antigenic determinant on an antigen to which the paratope of an antibody binds. Epitopic determinants usually consist of chemically active surface groupings of molecules such as amino acids or sugar side-chains and usually have specific three-dimensional structural characteristics, as well as specific charge characteristics. The unique amino acid sequence of the present sasp polypeptides provides a correspondingly unique epitope in three-dimensional space.

Monoclonal antibodies are made from antigen containing fragments of the unique polypeptide by methods well known in the art (Kohler, et al., Nature, 256:495, 1975; Current Protocols in Molecular Biology, Ausubel, et al., ed., 1989).

Monoclonal or polyclonal antibodies which bind to the sasp polypeptide of the invention can be prepared using an intact polypeptide or fragments containing small peptides of interest as the immunizing antigen. The polypeptide or a B. anthracis DNA sequence as shown in Example 6 and identified as NMRI#1, used to immunize an animal can be derived from translated cDNA or chemical synthesis which can be conjugated to a carrier protein, if desired. Such commonly used carriers, which are chemically coupled to the peptide, include keyhole limpet hemocyanin (KLH), thyroglobulin, bovine serum albumin (BSA), and tetanus toxoid. The coupled peptide is then used to immunize the animal (e.g., a mouse, a rat, or a rabbit).

If desired, polyclonal or monoclonal antibodies can be further purified, for example, by binding to and elution from a matrix to which the polypeptide, or a peptide to which the antibodies were raised, is bound. Those of skill in the art will know of various techniques common in the immunology arts for purification and/or concentration of polyclonal antibodies, as well as monoclonal antibodies (See for example, Coligan, et al., Unit 9, Current Protocols in Immunology, Wiley Interscience, 1991, incorporated by reference).

It is also possible to use the anti-idiotype technology to produce monoclonal antibodies which mimic an epitope. For example, an anti-idiotypic monoclonal antibody made to a first monoclonal antibody will have a binding domain in the hypervariable region which is the “image” of the epitope bound by the first monoclonal antibody. Thus, in the present invention, an anti-idiotype antibody produced from an antibody which binds to the synthetic peptide of the invention can bind to the site on B. anthracis sasp which forms the spore, thereby preventing sasp from participating in spore assembly.

Polynucleotide sequences encoding the polypeptide or synthetic peptide (B. anthracis DNA sequence shown in Example 6 and identified as NMRI#11) of the invention can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art. Such vectors are used to incorporate DNA sequences of the invention.

Accordingly, the present invention should be understood to encompass subject matter limited only by the scope of the appended claims.

What is claimed is:

1. A cloned and isolated nucleic acid encoding a Bacillus anthracis sasp-B protein having greater than 90% homology to the Bacillus cereus sasp-B DNA.

2. The nucleic acid of claim 1 having the nucleic acid sequence of SEQ ID NO: 87.

3. The nucleic acid of claim 1, further comprising the full length coding sequence for said sasp-B protein.

4. The nucleic acid of claim 1 having the nucleic acid sequence of SEQ ID NO: 107.

5. The nucleic acid of claim 1, wherein the nucleic acid encodes SEQ ID NO: 92.

6. An antibody that selectively binds to the Bacillus anthracis sasp-B protein.

7. The antibody of claim 6, wherein the Bacillus anthracis sasp-B protein has an amino acid sequence of SEQ ID NO: 92.

8. The antibody of claim 6, wherein the antibody binds to the epitope encoded by TAGCATT.

9. The antibody of claim 6, wherein said antibody is a monoclonal antibody.

10. A nucleic acid primer that hybridizes specifically to the sasp-B DNA of Bacillus anthracis.

11. A nucleic acid probe that hybridizes to the sequence 5′-TAG CAT T-3” or the complimentary strand thereof.

12. A method for the detection of Bacillus anthracis (B.a.) in a sample, comprising the steps of:
(a) incubating the sample with amplification primers that hybridize to the Bacillus anthracis sasp-B gene;
(b) amplifying a target sequence between the hybridized primers; and
(c) detecting the presence of amplified Bacillus anthracis sasp-B gene sequences.

13. The method of claim 12 comprising the steps of detecting the presence of an insert having SEQ ID NO: 107.

14. The method of claim 12, wherein the method of amplifying the sasp-B gene comprises the use of the polymerase chain reaction.

15. The method of claim 12, wherein the sasp-B gene primers hybridize to the forward and reverse strands of sequence of SEQ ID NO: 87.

16. The method of claim 12, wherein said detecting step comprises the step of hybridizing the amplified fragment to a probe specific for the Bacillus anthracis sasp-B gene.

17. A method for detecting the presence of Bacillus anthracis in a sample comprising the step of:
(a) incubating said sample with an antibody to the Bacillus anthracis sasp-B protein; and
(b) detecting the binding of said antibody to Bacillus anthracis sasp-B protein in the sample.

18. The method of claim 17, wherein said antibody is a monoclonal antibody.
